(19) United States
(12) Patent Application Publication
(10) Pub. No.: US 2009/0104711 A1
(43) Pub. Date: Apr. 23, 2009

(54) FLUORESCENT MARKER COMPRISING DOUBLE BOND ESTER GROUP AND METHOD FOR MARKING AND DETECTING THE SAME

(76) Inventor: Hyun-Ho Sim, Incheon (KR)

Correspondence Address: IPLA P.A., 3580 WILSHIRE BLVD., 17TH FLOOR, LOS ANGELES, CA, 90010, US

(21) Appl. No.: 12/064,623

(22) PCT Filed: Jul. 28, 2006
(86) PCT No.: PCT/KR2006/002968

§ 371 Date: Feb. 22, 2008

(30) Foreign Application Priority Data
Aug. 22, 2005 (KR) 10-2005-0076603

(51) Int. Cl. G01N 21/64; C07D 311/06; C07D 311/82; C07D 307/77; C07D 265/34

(52) U.S. Cl. 436/172; 549/283; 549/388; 549/344; 544/102; 549/285

(57) ABSTRACT

Disclosed herein are fluorescent markers having a double bond ester group and a method for marking and detecting the same. More particularly, disclosed are a method for identifying oil products, which comprises marking the oil products with fluorescent markers having an unsaturated double bond ester group, adding a developing agent having a function of inducing specific fluorescence to the marked oil products, and detecting the fluorescent marker with a fluorescence spectrophotometer in the UV/VIS region, as well as said markers.

### TECHNICAL FIELD

The present invention relates to fluorescent markers having a double bond ester group and a method for marking and detecting the same, and more particularly to a method for identifying oil products, which comprises marking the oil products with fluorescent markers having an unsaturated double bond ester group, adding a developing agent having a function of inducing specific fluorescence to the marked oil products, and detecting the fluorescent marker with a fluorescence spectrophotometer in the UV/VIS region.

### BACKGROUND ART

Petroleum markers have continued to be developed since the 1980s. Petroleum markers have been added and used for the following two reasons.

First, the petroleum markers have been used to prevent environmental pollution, and to prevent a reduction in the life cycle of vehicles from occurring due to the illegal use of petroleum oils, resulting from an abrupt increase in the price of crude oil. These have been introduced and used to prevent the use of imitation gasoline and the illegal use of tax-free oil for special purposes.

Second, due to the improvement of petroleum purification technology, resulting from the development of petrochemical technology, oil refining companies charge a great deal for quality competition and maintenance. For this reason, for branding and the conformation of maintenance of quality in oil refining companies, the petroleum markers have been introduced. These started to be added to gasoline at an initial stage, and the use thereof has been broadened to LPG and light oil.

Of these markers for addition to petroleum, coumarin alkylester (U.S. Pat. No. 5,980,593) and fluorescein alkylester (U.S. Pat. No. 5,498,808) are known. In these patents, saturated alkylesters are used as markers, and a basic extract or tetraalkylammonium is used as a color developing agent. In U.S. Pat. No. 5,980,593, coumarin as a starting material is introduced with a saturated alkylester group to prepare a gel or semi-solid material, which is then diluted. Also, in U.S. Pat. No. 5,498,808, a fluorescein derivative as a starting material is introduced with a saturated alkylester group to obtain a solid, waxy solid or oil for use as a marker, and tetraalkylammonium is used as a color developing agent. The tetraalkylammonium used in this US patent causes considerable undesirable sensations due to the unpleasant odor of ammonia during the use thereof. In addition to said fluorescent markers, it is known to use resorufin and other fluorescent materials (Korean Patent Publication No. 2002-6420) and to use lipase as a color developing agent. However, the fluorescent marker disclosed in said Korean Patent Publication needs to be improved, because it requires complex analytical processes or expensive reagents.

### DISCLOSURE OF INVENTION

### Technical Problem

Accordingly, the present invention has been made to solve the above-described problems of the prior fluorescent markers. An object of the present invention is to provide fluorescent markers suitable for the identification of oil products, which do not cause an unpleasant odor, allow an inexpensive developing agent to be used and require a simple analytical process.

Another object of the present invention is to provide a method for identifying oil products, which comprises marking the oil products with said fluorescent markers and detecting the fluorescent markers in the oil products.

### Technical Solution

To achieve the above objects, the present invention provides novel fluorescent markers represented by Formulas 1 to 3:

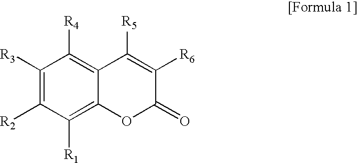

wherein R₁, R₃, R₄ and R₆ each independently represent hydrogen, a nitro group, a halogen atom, a carboxyl group, an amino group, a substituted or unsubstituted C1-C30 alkyl group, a substituted or unsubstituted C2-C30 alkenyl group, a substituted or unsubstituted C6-C30 aryl group, a substituted or unsubstituted C1-C30 heteroalkyl group, a substituted or unsubstituted C2-C30 heteroalkenyl group or a substituted or unsubstituted C4-C30 heteroaryl group;

R₂ and R₅ each independently represent hydrogen, a nitro group, a halogen atom, a carboxyl group, an amino group, a substituted or unsubstituted C1-C30 alkyl group, a substituted or unsubstituted C2-C30 alkenyl group, a substituted or unsubstituted C6-C30 aryl group, a substituted or unsubstituted C1-C30 heteroalkyl group, a substituted or unsubstituted C2-C30 heteroalkenyl group, a substituted or unsubstituted C4-C30 heteroaryl group or —OC(═O)—R₇,

wherein R₇ represents a substituted or unsubstituted C2-C30 alkenyl group or a substituted or unsubstituted C2-C30 heteroalkenyl group, with the proviso that at least one R₂ and R₅ represents —OC(═O)—R₇;

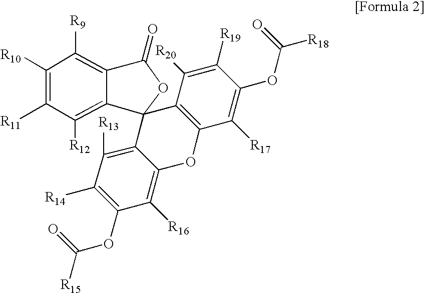

wherein R₉ to R₂₀ each independently represent hydrogen, a nitro group, a halogen atom, a carboxyl group, an amino group, a substituted or unsubstituted C1-C30 alkyl group, a substituted or unsubstituted C2-C30 alkenyl group, a substituted or unsubstituted C6-C30 aryl group, a substituted or unsubstituted C1-C30 heteroalkyl group, a substituted or unsubstituted C2-C30 heteroalkenyl group, or a substituted or unsubstituted C4-C30 heteroaryl group, with the proviso that at least one of R₁₅ and R₁₈ represents a substituted or unsubstituted C2-C30 alkenyl group or a substituted or unsubstituted C2-C30 heteroalkenyl group; and

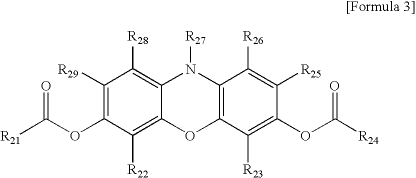

wherein R₂₁ to R₂₉ each independently represent hydrogen, a nitro group, a halogen atom, a carboxyl group, an amino group, a substituted or unsubstituted C1-C30 alkyl group, a substituted or unsubstituted C2-C30 alkenyl group, a substituted or unsubstituted C6-C30 aryl group, a substituted or unsubstituted C1-C30 heteroaryl group, a substituted or unsubstituted C2-C30 heteroalkenyl group, or a substituted or unsubstituted C4-C30 heteroaryl group, with the proviso that at least one of R₂₁ and R₂₄ represents a substituted or unsubstituted C2-C30 alkenyl group or a substituted or unsubstituted C2-C30 heteroalkenyl group.

As used in said compounds according to the present invention, the term “alkyl group” embraces a straight- or branched-chain alkyl radical having 1 to 30 carbon atoms, and preferably a straight or branched-chain alkyl radical having 1 to about 12 carbon atoms. A more preferred alkyl radical is a lower alkyl radical having 1 to 6 carbon atoms. Examples of this alkyl radical may include methyl, ethyl, n-propyl, isopropyl, n-butyl, isobutyl, sec-butyl, t-butyl, pentyl, iso-amyl, hexyl and the like. Still more preferred is a lower alkyl radical having 1 to 3 carbon atoms.

As used in said compounds according to the present invention, the term “alkenyl group” means a straight- or branched-chain aliphatic unsaturated hydrocarbon group having 2 to 30 carbon atoms and containing a carbon-carbon double bond. A preferred alkenyl group has 1 to 20 carbon atoms, and more preferably 4 to 18 carbon atoms, in the chain. The branched chain means that at least one lower alkyl or lower alkenyl group is attached to a straight alkenyl chain. This alkenyl group is unsubstituted or may be independently substituted with at least one group selected from the group consisting of halo, carboxyl, hydroxyl, formyl, sulfo, sulfino, carbamoyl, amino and imino groups, but is not limited thereto. Examples of this alkenyl group include ethenyl, prophenyl, carboxyethenyl, carboxypropenyl, sulfinoethenyl, sulfonoethenyl, and the like.

As used in said compounds according to the present invention, the term “aryl group”, alone or in combination, means a C6-C20 carbocyclic aromatic system containing one or more rings, wherein such rings may be bonded together in a pendent manner or may be fused. The term “aryl” embraces aromatic radicals such as phenyl, naphthyl, tetrahydronaphthyl, indane and biphenyl. A more preferred aryl is phenyl. The aryl group may have 1 to 3 substituents such as hydroxyl, halo, haloalkyl, nitro, cyano, alkoxy and lower alkylamino.

As used in said compounds according to the present invention, the term “heteroalkyl group” refers to an alkyl group, as defined above, wherein one or more carbon atoms in the chain are substituted with one, two or three heteroatoms selected from among N, O and S.

As used in said compounds according to the present invention, the term “heteroalkenyl group” refers to an alkenyl group, as defined above, wherein one or more carbon atoms in the chain are substituted with one, two or three heteroatoms selected from among N, O and S.

As used in said compounds according to the present invention, the term “heteroaryl group” refers to a monovalent monocyclic or bicyclic aromatic radical of 6 to 20 ring atoms containing one, two or three heteroatoms selected from N, O and S, the remaining ring atoms being C. Also, the term refers to a monovalent monocyclic or bicyclic aromatic radical, in which the heteroatom in the ring is oxidized or quaternarized to form, for example, N-oxide or quaternary salt. Typical examples thereof include thienyl, benzothienyl, pyridyl, pyrazinyl, pyrimidinyl, pyridazinyl, quinolinyl, quinoxalinyl, imidazolyl, furanyl, benzofuranyl, thiazolyl, isoxazolyl, benzisoxazolyl, benzimidazolyl, triazolyl, pyrazolyl, pyrrolyl, indolyl, 2-pyridonyl, 4-pyridonyl, N-alkyl-2-pyridonyl, pyrazinonyl, pyridazinonyl, pyrimidinonyl, oxazolonyl, and their corresponding N-oxides (e.g. pyridyl N-oxide, quinolinyl N-oxide), their quaternary salts and the like.

The fluorescent markers of Formulas 1 to 3 according to the present invention have an increased solubility in oil products, allow an inexpensive developing agent to be used and, at the same time, do not cause an unpleasant odor.

Typical fluorescent compounds are polar in nature, and thus have a poor solubility in conventional non-polar oil products. For this reason, in order to use these compounds as markers for conventional oil products, the introduction of a non-polar group into the compounds is necessary. In the present invention, the solubility of the fluorescent compounds in oil products can be improved by introducing an ester group having at least one unsaturated double bond into the fluorescent compounds so as to enhance the non-polarity of the fluorescent compounds. Also, the fluorescent markers do not emit strongly volatile substances such as ammonia, and thus do not cause unpleasant sensations. In addition, these markers have an increased solubility in oil products, and thus eliminate a need to use an expensive solvent, leading to an improvement in economic efficiency.

The compounds of Formulas 1 to 3 can be prepared by introducing an unsaturated double bond ester group into coumarin, fluorescein and resorufin compounds as starting materials, but are not limited thereto and can be prepared using any conventional method known in the art. This preparation can be achieved by introducing an unsaturated double bond into said starting materials, considering that the melting point of unsaturated alkyl acid derivatives is lower than that of saturated alkyl acid derivatives used for the introduction of non-polar groups in the prior art (e.g. the melting point of stearic anhydride: 70-72° C., and the melting point of oleic anhydride: 22-24° C.).

The coumarin-based compound of Formula 1 can be exemplified by a compound represented by Formulas 4 and 5:

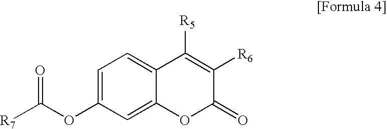

wherein R₅ to R₇ are defined the same way as above;

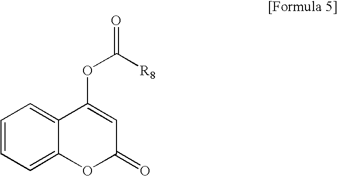

wherein R₈ represents a substituted or unsubstituted C2-C30 alkenyl group or a substituted or unsubstituted C2-C30 heteroalkenyl group.

The fluorescein-based compound of Formula 2 is preferably a compound represented by Formula 6:

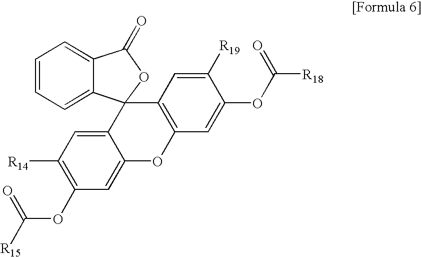

wherein R₁₄, R₁₅, R₁₈ and R₁₉ are defined the same way as above.

The resorufin-based compound of Formula 3 is preferably a compound represented by Formula 7:

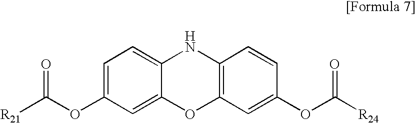

wherein R₂₁ and R₂₄ are defined the same way as above.

If the functional group substituted into the ester group in the compounds of Formulas 1 to 7 according to the present invention is an alkenyl group having an unsaturated double bond, examples thereof include an acryl group, methacryl group, crotonyl group, hexenoyl group, octenoyl group, oleoyl group and the like, the preferred being an oleoyl group.

The fluorescent markers of Formulas 1 to 3 according to the present invention can be added to various oil products, for example, gasoline, light oil, fuel oil, kerosene and burning-oil. More specifically, these markers can be added to gasoline for vehicles, liquefied natural gas or liquefied petroleum gas for vehicles, or heavy oil for various motors. In addition, these markers can be added to petroleum (burning-oil) for fuel, liquefied natural gas for fuel, liquefied petroleum gas, and important fuel products.

Thus, the use of the fluorescent markers according to the present invention allows the identification of all oil products, including imitation gasoline prepared by adding chemicals such as benzene, toluene and solvents, as well as gasoline produced and supplied by persons other than legal suppliers, including owners of trademarks of oil products, and also various motor oils and fuel oils.

In another aspect, the present invention provides a method for identifying oil products, comprising the steps of: (1) marking the oil products with at least one of the fluorescent markers of Formulas 1 to 3; and (2) adding an alkanol metal salt as a developing agent to the marked oil products to detect fluorescence.

In said step (1), the oil products are marked with the fluorescent markers of the present invention.

The fluorescent markers of Formulas 1 to 3 according to the present invention have little or no effect on the performance of the oil products, because these are used in very small amounts in the oil products. Also, since the inventive fluorescent markers have high solubility in these oil products, these have little or no possibility to form sludge or a precipitate during the use of the oil products, and thus cause little or no damage to machines and systems, which use these oil products as fuel.

The fluorescent markers according to the present invention can be detected in oil products even when the content thereof in the oil products is very low. Thus, the fluorescent markers according to the present invention are added to oil products in an amount of 0.01-100 ppm, preferably 0.01-30 ppm, and more preferably 0.01-10 ppm. If the content of the markers in the oil products is less than 0.01 ppm, the markers can be difficult to detect because the development of fluorescence by the developing agent is low. On the other hand, a fluorescent marker content exceeding 100 ppm is economically undesirable, because the increase in the marker content does not bring about any beneficial color development

Meanwhile, the inventive markers emit fluorescent light having different wavelengths depending on the type of the substituents. Thus, these markers can be used alone or in combinations of two or more thereof. These markers can be added to oil products in the form of solids such as powders or crystals or in the form of a liquid such as a concentrate. These are preferably used in the form of a liquid in view of handling problems and the like.

To prepare a liquid concentrate containing the marker according to the present invention, the marker can be dissolved or diluted in an organic solvent to prepare a non-aqueous solution having a high solubility in oil products. Solvents suitable for use with liquid petroleum products are generally aromatic and aprotic solvents. Preferred examples thereof, which can be used in the present invention, include aromatic hydrocarbons (e.g., alkylbenzene such as xylene and naphthalene), aromatic alcohols (e.g., benzylalcohol) and aromatic substituted alkanols (e.g., phenolglycolether). The aprotic solvents include formamide, N,N-dimethylformamide, N,N-dimethylacetamide, 1-methylpyrrolidinone, 1-octyl pyrrolidinone, 1-dodecyl pyrrolidinone and the like. 1-octyl pyrrolidinone is a particularly preferred aprotic solvent. Such solvents can be used alone or in combination.

Then, in step (2), an alkanol metal salt as a color-developing agent is added to the marked oil products to detect fluorescence.

The markers according to the present invention are colorless or substantially colorless and are soluble in oil products. Thus, quantification and qualification are possible by adding the developing agent to the oil products marked with the markers to allow the developing agent to react with the markers and measuring the fluorescence of the oil products. As the alkanol metal salt, an alkanol metal salt enabling the fluorescence of all oil products to be measured, for example, a C1-C10 alkanol lithium salt, alkanol sodium salt and alkanol potassium salt, is preferably used in the present invention. Examples of the alkanol may include methanol, ethanol, butanol, t-butanol, and 2-ethylhexanol. Particularly preferred are sodium methoxide, lithium methoxide, potassium methoxide and the like.

The developing agent such as the alkanol metal salt hydrolyzes ester bonds in the markers according to the present invention so as to enable the markers to be detected with a fluorescence spectrophotometer. The marked oil products can be quantitatively and qualitatively analyzed for the presence or absence and concentrations of the markers by detecting fluorescence occurring in the UV/VIS region.

A process for the development of fluorescence using the developing agent will now be described in detail.

The fluorescent markers of Formulas 1 to 3 according to the present invention are colorless or substantially colorless before the addition of the developing agent, and when a given amount of the developing agent according to the present invention is added to oil products marked with the markers, the markers of Formulas 1 to 3 will be converted into fluorescent anions. For example, the ester group of the markers, which has an unsaturated double bond, is hydrolyzed by the developing agent base. Then, when a specific wavelength of light is irradiated into the oil products, the oil products will have strong fluorescence. The fluorescent markers of Formulas 1 to 3 are not easily extracted from organic media such as oil products, whereas the development pattern of the fluorescence of the markers represented by Formulas 1 to 3 allows the analysis of the markers, the fluorescence of which was developed in the oil products.

Accordingly, the markers of Formulas 1 to 3 according to the present invention can be quantitatively analyzed, and thus, if an imitation gasoline product is prepared by diluting gasoline in a solvent such as toluene or benzene, it can easily be determined whether it is genuine using the inventive markers. In other words, if a given concentration of the markers is added to an oil product, and then, if the markers in the oil product are quantitatively detected at a concentration lower than the original concentration thereof, the oil product can be easily determined to be diluted.

### BRIEF DESCRIPTION OF THE DRAWINGS

FIG. 1 is a graphic diagram showing the results of quantitative analysis for petroleum marked with 7-coumarin oleate of Example 2 according to the present invention.

FIG. 2 is a graphic diagram showing the results of quantitative analysis for petroleum marked with fluorescein dioleate of Example 6 according to the present invention.

FIG. 3 is a graphic diagram showing the results of quantitative analysis for petroleum marked with resorufin dioleate of Example 9 according to the present invention.

FIG. 4 is a photograph showing the comparison of solubility between the compound of Example 6 and a C18 saturated alkyl-chain ester (fluorescein distearate): (1) unsaturated ester marker (Example 6: fluorescein dioleate); (2) saturated ester marker (fluorescein distearate); (3) the result after solubilizing in petroleum (left: (1), right: (2)).

### MODE FOR THE INVENTION

Hereinafter, the present invention will be described in further detail with reference to examples. It is to be understood, however, that these examples are for illustrative purposes and are not to be construed to limit the scope of the present invention.

### Example 1

### Synthesis of Coumarin Oleate

Into a 100-ml three-neck reactor, 1.43 g of 4-hydroxycoumarin, 30 ml of methylene chloride and 1.07 g of triethylamine were added, and the mixture was stirred at a temperature of less than 7° C. for 30 minutes. To the stirred mixture, 5 ml of 70% oleoyl chloride were added dropwise over 1 hour using a dropping funnel. Then, the mixture was maintained below 10° C. for 3 hours and then left to stand at room temperature for 24 hours, after which 50 ml of distilled water were added thereto. Then, the solution was separated into layers. To the collected organic layer, 2 ml of 1 N hydrochloric acid were added, and the solution was separated into layers. The resulting organic layer was neutralized with a 5% sodium carbonate solution. The neutralized organic layer was dried with anhydrous magnesium sulfate, followed by filtration. After removing the methylene chloride of the organic layer under reduced pressure, the residue was concentrated in a vacuum, yielding 3.62 g (96.2% yield) of 4-coumarin oleate as yellow liquid.

¹H NMR (400 MHz, CDCl₃): δ 0.886 (m, 3H, CH₃), 1.29-1.45 (m, 22H, CH₂), 2.01 (m, 4H, CH₂ (allyl)), 2.70 (t, 2H, CH₂), 5.31-5.39 (m, 2H, CH═CH), 6.65 (s, 1H, pyranone), 7.30 (d, 1H, aromatic), 7.34-7.58 (m, 2H, aromatic), 7.66 (d, 1H, aromatic)

IR (cm⁻¹): 2924, 1735, 1627, 1102

### Example 2

### Synthesis of 7-coumarin Oleate

Into a 100-ml three-neck reactor, 1.25 g of 7-hydroxycoumarin, 0.85 g of triethylamine and 30 ml of methylene chloride were added, and the mixture was stirred while maintaining 5° C. To the stirred mixture, 4 ml of 70% oleoyl chloride and 10 ml of methylene chloride were added dropwise over 1 hour using a dropping funnel. Then, the mixture was maintained below 10° C. for 3 hours and then at room temperature for 18 hours, after which 50 ml of distilled water were then added thereto. Then, the solution was separated into layers. To the collected organic layer, 4 ml of 1 N hydrochloric acid were added and the solution was separated into layers. The resulting organic layer was neutralized with a 5% sodium carbonate solution. The neutralized organic layer was dried with anhydrous magnesium sulfate, followed by filtration. After removing the methylene chloride of the organic layer under reduced pressure, the residue was concentrated in a vacuum, yielding 2.92 g (89.0% yield) of 7-coumarin oleate as the desired product.

¹H NMR (400 MHz, CDCl₃): δ 0.878 (m, 3H, CH₃), 1.28-1.43 (m, 22H, CH₂), 2.04 (m, 4H, CH₂ (allyl)), 2.60 (t, 2H, CH₂), 5.3-5.40 (m, 2H, CH═CH), 6.38-6.41 (d, 1H, pyranone), 7.04 (d, 1H, pyranone), 7.06 (s, 1H, aromatic), 7.48-7.72 (m, 2H, aromatic)

IR (cm⁻¹): 2920, 1747, 1627, 1564, 1122

### Example 3

### Synthesis of 6-chloro-4-coumarin Oleate

Into a 100-ml three-neck flask, 0.4 g of 6-chloro-4-hydroxycoumarin, 0.25 g of triethylamine and 30 ml of methylene chloride were added, and the mixture was stirred while maintaining a temperature of 5° C. To the stirred mixture, 1.2 ml of 70% oleoyl chloride and 10 ml of methylene chloride were added dropwise over 1 hour using a dropping funnel. Then, the same process as in Example 2 was carried out, thus obtaining 0.89 g (95.1% yield) of 6-chloro-4-coumarin oleate as the desired product.

¹H NMR (400 MHZ, DMSO): δ 0.85 (m, 3H, CH₃), 1.23-1.49 (m, 22H, CH₂), 2.02 (m, 4H, CH₂ (allyl)), 2.50 (t, 2H, CH₂), 5.3-5.33 (m, 2H, CH═CH), 5.60 (s, 1H, pyranone), 7.41-7.69 (d, 2H, aromatic), 7.77 (s, 1H, aromatic)

IR (cm⁻¹): 2927, 1704, 1623, 1568, 1122

### Example 4

### Synthesis of 4-methyl-7-coumarin Oleate

Into a 100-ml three-neck flask, 1.55 g of 4-methyl-7-hydroxycoumarin, 1.07 g of triethylamine and 30 ml of methylene chloride were added, and the mixture was stirred while maintaining a temperature of 5° C. To the stirred mixture, 5 ml of 70% oleoyl chloride and 10 ml of methylene chloride were added dropwise over 1 hour using a dropping funnel. Then, the same process as in Example 2 was carried out, thus obtaining 3.81 g (98.2% yield) of 4-methyl-7-coumarin oleate as the desired product.

¹H NMR (400 MHz, CDCl₃): δ 0.89 (m, 3H, CH₃), 1.30-1.65 (m, 22H, CH₂), 2.04 (m, 4H, CH₂ (allyl)), 2.46 (s, 3H, pyranone), 2.62 (t, 2H, CH₂), 5.35-5.42 (m, 2H, CH═CH), 6.31 (s, 1H, pyranone), 7.09 (d, 1H, aromatic), 7.11 (s, 1H, aromatic), 7.62 (d, 1H, aromatic)

IR (cm⁻¹): 2927, 1712, 1619, 1460, 1133

### Example 5

### Synthesis of Fluorescein Dimethacrylate

2.63 g of fluorescein, 1.52 g of triethylamine and 30 ml of methylene chloride were added into a reactor and stirred at a temperature of less than 7° C. for 30 minutes. 2 ml of 80% methacryl chloride were added dropwise thereto at a temperature of less than 8° C. over 1 hour. Then, the same process as in Example 2 was carried out, thus obtaining 3.11 g (88.4% yield) of fluorescein dimethacrylate as the desired product.

¹H NMR (400 MHz, DMSO): δ 2.0 (d, 6H, CH₃), 5.92-6.30 (m, 4H, CH═CH (acryl)), 6.60 (d, 2H, xanthene), 6.82 (d, 2H, xanthene), 6.92 (s, 2H, xanthene), 7.34 (m, 1H, aromatic), 7.77-7.81 (m, 2H, aromatic), 8.01 (m, 1H, aromatic)

IR (cm⁻¹): 2974, 1739, 1603, 1463, 1122

### Example 6

### Synthesis of Fluorescein Dioleate

1.3 g of fluorescein, 0.85 g of triethylamine and 30 ml of methylene chloride were added into a reactor and stirred at a temperature of less than 7° C. for 30 minutes. 4 ml of 70% oleoyl chloride were added dropwise thereto at a temperature of less than 8° C. over 1 hour. Then, the same process as in Example 2 was carried out, thus obtaining 3.1 g (93.8% yield) of fluorescein dioleate as the desired product.

¹H NMR (400 MHz, CDCl₃): δ 0.90 (m, 6H, CH₃), 1.30-1.37 (m, 44H, CH₂), 2.03 (m, 4H, CH₂ (allyl)), 5.37-5.42 (m, 4H, CH═CH), 6.82-6.87 (d, 4H, xanthene), 7.12 (s, 2H, xanthene), 7.22 (m, 1H, aromatic), 7.66 (m, 1H, aromatic), 7.72 (m, 1H, aromatic), 8.08 (m, 1H, aromatic)

IR (cm⁻¹): 2920, 1770, 1611, 1467, 1161

### Example 7

### Synthesis of 2,7-dichlorofluorescein Dioleate

1.55 g of 2,7-dichlorofluorescein, 0.86 g of triethylamine and 30 ml of methylene chloride were added into a reactor and stirred at a temperature of less than 7° C. for 30 minutes. 4 ml of 70% oleoyl chloride were added dropwise thereto at a temperature of less than 8° C. over 1 hour. Then, the same process as in Example 2 was carried out, thus obtaining 2.84 g (79.5% yield) of 2,7-dichlorofluorescein dioleate as the desired product.

¹H NMR (400 MHz, CDCl₃): δ 0.88 (m, 6H, CH₃), 1.30-1.36 (m, 44H, CH₂), 2.02 (m, 4H, CH₂ (allyl)), 2.64 (t, 4H, CH₂), 5.37 (s, 4H, CH═CH), 6.90 (s, 2H, xanthene), 7.15 (s, 2H, xanthene), 7.22 (d, 1H, aromatic), 7.69-7.72 (m, 2H, aromatic), 8.08 (d, 1H, aromatic)

IR (cm⁻¹): 2931, 1766, 1599, 1471, 1172

### Example 8

### Synthesis of Resorufin Dicrotonylate

0.4 g of resorufin, 0.42 g of triethylamine and 30 ml of methylene chloride were added into a reactor and stirred at a temperature of less than 7° C. for 30 minutes. 0.44 ml of 90% crotonyl chloride were added dropwise thereto at a temperature of less than 8° C. over 1 hour. Then, the same process as in Example 2 was carried out, thus obtaining 0.39 g (60.0% yield) of resorufin dicrotonylate as the desired product.

¹H NMR (400 MHz, CDCl₃): δ 1.96 (m, 6H, CH₃), 5.87-5.91 (m, 4H, CH═CH), 6.96 (m, 2H, aromatic), 7.10-7.16 (m, 2H, aromatic), 7.30 (s, 2H, aromatic)

IR (cm⁻¹): 2920, 1716, 1603, 1561, 1176

### Example 9

### Synthesis of Resorufin Dioleate

0.5 g of resorufin, 0.52 g of triethylamine and 30 ml of methylene chloride were added into a reactor and stirred at a temperature of less than 7° C. for 30 minutes. 2.4 ml of 70% oleoyl chloride were added dropwise thereto at a temperature of less than 8° C. over 1 hour. Then, the same process as in Example 2 was carried out, thus obtaining 1.27 g (73.0% yield) of resorufin dioleate as the desired product.

¹H NMR (400 MHz, CDCl₃): δ 0.913 (m, 6H, CH₃), 1.32-1.36 (m, 44H, CH₂), 2.02 (m, 4H, CH₂ (allyl)), 5.38-5.40 (m, 4H, CH═CH), 6.92 (m, 2H, aromatic), 7.17-7.19 (m, 2H, aromatic), 7.21 (s, 2H, aromatic)

IR (cm⁻¹): 2920, 1743, 1650, 1440, 1153

### Example 10

### Qualitative Analysis Using Markers

Each of the compounds prepared in Examples 1, 2, 4, 6, 7 and 9 was diluted to a concentration of 40% by adding Aromatic 150 (produced by SK Corporation, Korea) thereto, and each of the compounds prepared in Examples 3, 5 and 8 was added to the same amount of 1-octylpyrrolidone and diluted to a concentration of 20% by adding Aromatic 150 (produced by SK Corporation, Korea) thereto. Each of these marker solutions was added to kerosene oil at a concentration of 10 ppm, and 50 ml of each of the oils were taken, to which 5 ml of 7.5% lithium methoxide were then added. Then, each of the oils was measured for fluorescence using an ISS K2 fluorescence spectrophotometer, and the measurement results are shown in Table 1 below.

TABLE 1

| | Example | Example | Example | Example | Example | Example | Example | Example | Example |
|---|---|---|---|---|---|---|---|---|---|
| | 1 | 2 | 3 | 4 | 5 | 6 | 7 | 8 | 9 |
| λₘₐₓ ₍ₙₘ₎ | 471 | 460 | 460 | 460 | 525 | 523 | 530 | 572 | 572 |
| Irradiation wavelength | 365 | 365 | 350 | 365 | 490 | 490 | 490 | 570 | 570 |
| (nm) | | | | | | | | | |
| Color | blue | blue | blue | blue | green | green | Reddish | red | red |
| | | | | | | | green | | |

Also, it was observed that the coumarin, the fluorescein and the resorufin showed fluorescence having the colors of blue, green and red, respectively, when irradiated with a UV lamp of an appropriate wavelength. As can be seen in the results shown in Table 1, the qualitative analysis of the oils using the markers of the present invention is possible.

### Example 11

### Quantitative Analysis Using Markers

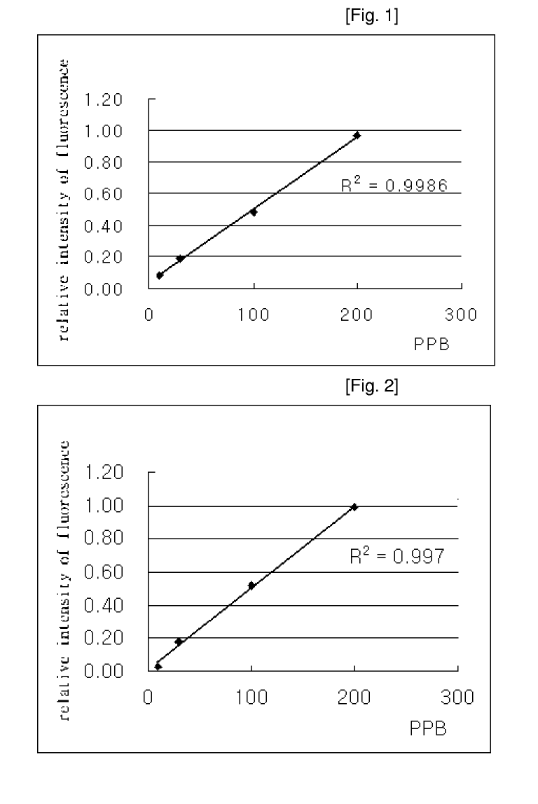

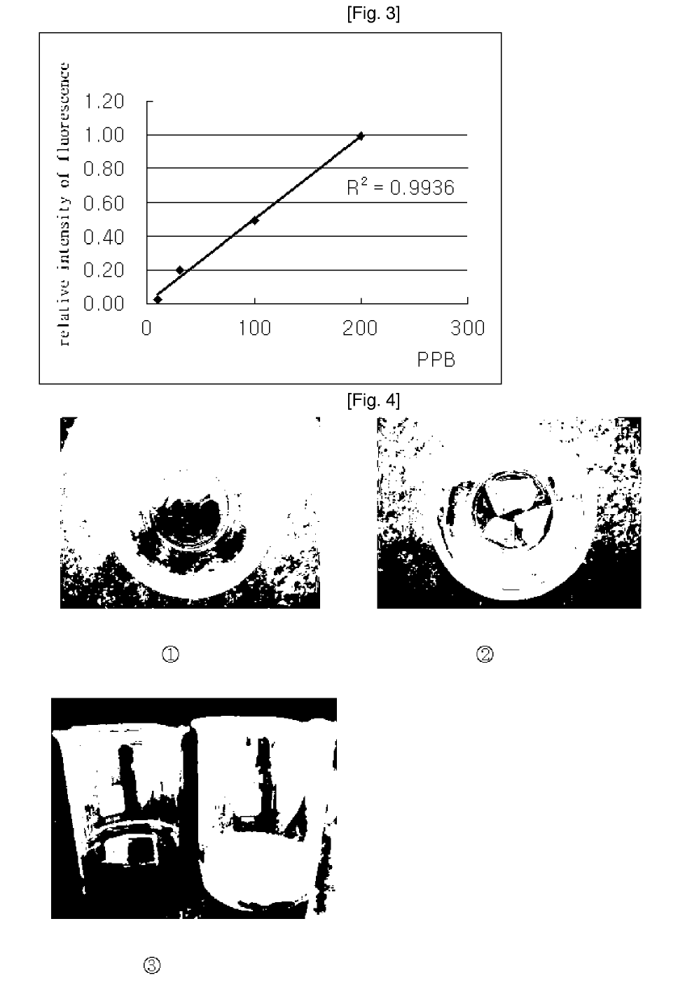

Each of the compounds prepared in Examples 2, 6 and 9 was diluted to a concentration of 40% by adding an Aromatic 150 solvent thereto. Each of these marker solutions was added to petroleum oil at concentrations of 10, 30, 100 and 200 ppb, and 50 ml of each of the petroleum oils was taken, to which 5 ml of 10% sodium methoxide solution (a mixture of methanol and 2-ethyl hexanol) were then added. Then, each of the oils was analyzed with a fluorescence spectrophotometer, and the measurement results are shown in FIGS. 1 to 3.

As can be seen in FIGS. 1 to 3, when the markers and developing agent according to the present invention are used in oil products, they can easily achieve the qualitative and quantitative analysis of the oil products due to the high solubility thereof.

FIG. 4 is a photograph showing the comparison of solubility between the compound of Example 6 and a C18 saturated alkyl-chain ester (fluorescein distearate). As can be seen in FIG. 4, there is a significant difference in solubility between the compound of Example 6 and the C18 saturated alkyl chain ester (fluorescein distearate).

Referring to Table 2 below, it is evident that the solubility of unsaturated esters in kerosene oil is significantly higher than that of saturated esters. For reference, for relative comparison with saturated alkyl esters (C16 palmitates) disclosed in, for example, WO 02/04431, C18 stearates, which are the same alkyl chain as the C16 palmitates, were used for comparison with the inventive compounds.

TABLE 2

| | | | Rate of increase in | Rate of increase in |
|---|---|---|---|---|
| | | | fluorescent intensity of | solubility of |
| Examples | Substances | Solubility (%) | unsaturated esters (%) | unsaturated esters (%) |
| Example 4 | 4-methyl-7-coumarin oleate | More than 14.7 | More than 20,000 | More than 2,000 |
| Comparative | 4-methyl-7-coumarin stearate | 0.57 | | |
| Example | | | | |
| Example 6 | Fluorescein dioleate | More than 14.7 | More than 517 | More than 150 |
| Comparative | Fluorescein distearate | 9.8 | | |
| Example | | | | |
| Example 9 | Resorufin dioleate | More than 14.7 | More than 900 | More than 282 |
| Comparative | Resorufin distearate | 5.2 | | |
| Example | | | | |

As can be seen from the results shown in Table 2 above, the solubility of the fluorescein-based and resorufin-based compounds is higher than that of the coumarin-based compounds. This increase in the solubility of the diesters compared to the monoesters is believed to be attributable to the alkenyl ester branch of the diesters. Although the coumarin-based compounds have been examined and used as petroleum markers, the improvement in the solubility thereof, which has been pointed out as the inherent shortcoming thereof, can be ensured by introducing a double bond ester group into the molecular structure thereof. As can be seen from the results in Table 2 above, the increase in the solubility of the markers is more greatly influenced by factors resulting from the inclusion or non-inclusion of the double bond than the number of esters. It is to be understood that, because liquid substances have a solubility higher than that of solid substances, the fluorescent intensity of the liquid substances is also higher in proportion to the solubility thereof. For reference, the liquid substances prepared in Examples 4, 6 and 9 have a significantly high solubility of at least 14.7.

### INDUSTRIAL APPLICABILITY

As described above, the present invention provides the method for identifying oil products using, as markers, fluorescent ester substances having an unsaturated double bond, and as a developing agent, an alkanol metal salt. The markers are obtained by introducing unsaturated double bond esters into phthalein derivatives, in which the unsaturated ester can increase the solubility of the markers in apolar solvents, compared to saturated esters. Also, according to the present invention, the alkanol metal salt as the developing agent is used to develop fluorescence in an entire petroleum layer, and thus the unpleasant odor of ammonia, which occurs upon the use of the prior developing agent, can be eliminated.

Having described the present invention with reference to particular compositions, theories of effectiveness and the like, it will be apparent to those of skill in the art that it is not intended that the present invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence that is effective to meet the objectives intended, unless the context specifically indicates the contrary.

1. A fluorescent marker represented by Formula 1: 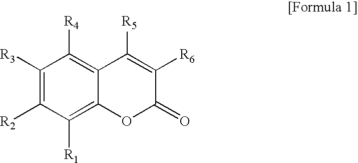
wherein R₁, R₃, R₄ and R₆ each independently represent a hydrogen atom, a nitro group, a halogen atom, a carboxyl group, an amino group, a substituted or unsubstituted C1-C30 alkyl group, a substituted or unsubstituted C2-C30 alkenyl group, a substituted or unsubstituted C6-C30 aryl group, a substituted or unsubstituted C1-C30 heteroalkyl group, a substituted or unsubstituted C2-C30 heteroalkenyl group or a substituted or unsubstituted C4-C30 heteroaryl group; and R₂ and R₅ each independently represent a hydrogen atom, a nitro group, a halogen atom, a carboxyl group, an amino group, a substituted or unsubstituted C1-C30 alkyl group, a substituted or unsubstituted C2-C30 alkenyl group, a substituted or unsubstituted C6-C30 aryl group, a substituted or unsubstituted C1-C30 heteroalkyl group, a substituted or unsubstituted C2-C30 heteroalkenyl group, a substituted or unsubstituted C4-C30 heteroaryl group or —OC(═O)—R₇, wherein R₇ represents a substituted or unsubstituted C2-C30 alkenyl group or a substituted or unsubstituted C2-C30 heteroalkenyl group, with the proviso that at least one R₂ and R₅ represents —OC(═O)—R₇.

2. A fluorescent marker represented by Formula 2: 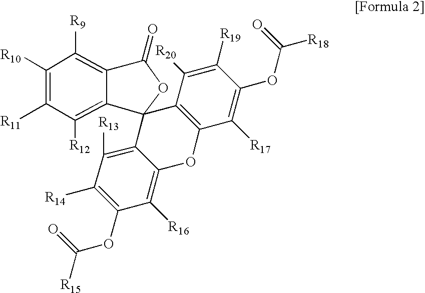
wherein R₉ to R₂₀ each independently represent a hydrogen atom, a nitro group, a halogen atom, a carboxyl group, an amino group, a substituted or unsubstituted C1-C30 alkyl group, a substituted or unsubstituted C2-C30 alkenyl group, a substituted or unsubstituted C6-C30 aryl a substituted C1-C30 heteroaryl group, a substituted or unsubstituted C2-C30 heteroalkenyl group, or a substituted or unsubstituted C4-C30 heteroaryl group, with the proviso that at least one of R₁₅ and R₁₈ represents a substituted or unsubstituted C2-C30 alkenyl group or a substituted or unsubstituted C2-C30 heteroalkenyl group.

3. A fluorescent marker represented by Formula 3: 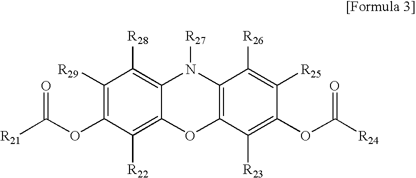
wherein R₂₁ to R₂₉ each independently represent a hydrogen atom, a nitro group, a halogen atom, a carboxyl group, an amino group a substituted or unsubstituted C1-C30 alkyl group, a substituted or unsubstituted C2-C30 alkenyl group, a substituted or unsubstituted C6-C30 aryl group, a substituted or unsubstituted C1-C30 heteroalkyl group, a substituted or unsubstituted C2-C30 heteroalkenyl group, or a substituted or unsubstituted C4-C30 heteroaryl group, with the proviso that at least one of R₂₁ and R₂₄ represents a substituted or unsubstituted C2-C30 alkenyl group or a substituted or unsubstituted C2-C30 heteroalkenyl group.

4. The fluorescent marker of claim 1, which is a compound represented by Formula 4 or 5: 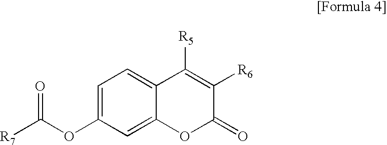
wherein R₅ to R₇ are defined the same way as in claim 1; and 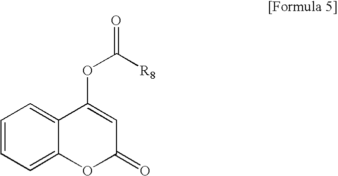
wherein R₈ represents a substituted or unsubstituted C2-C30 alkenyl group or a substituted or unsubstituted C2-C30 heteroalkenyl group.

5. The fluorescent marker of claim 2, which is a compound represented by Formula 6: 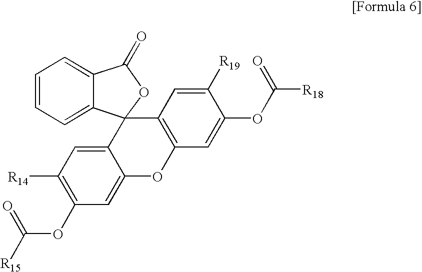
wherein R₁₄, R₁₅, R₁₈ and R₁₉ are defined the same way as in claim 2.

6. The fluorescent marker of claim 3, which is a compound represented by Formula 7: 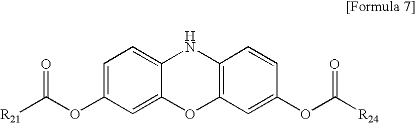
wherein R₂₁ and R₂₄ are defined the same way as in claim 3.

7. A method for identifying oil products, comprising the steps of:
marking the oil products with the fluorescent marker according to claim 1; and
detecting fluorescence in the marked oil products using a developing agent.

8. The method of claim 7, wherein the concentration of the fluorescent marker in the oil products is 0.01-100 ppm.

9. The method of claim 7, wherein the developing agent is an alkanol metal salt.
